(19) United States
(12) Patent Application Publication
(10) Pub. No.: US 2023/0069450 A1
(43) Pub. Date: Mar. 2, 2023

(54) PIGMENT SUSPENSION AND COSMETIC AGENT PREPARED BY USING THE PIGMENT SUSPENSION

(71) Applicant: Henkel AG & Co. KGaA, Duesseldorf (DE)

(72) Inventors: Rene KROHN, Norderstedt (DE); Thomas HIPPE, Appen (DE); Jessica BRENDER, Hamburg (DE); Stefan HOEPFNER, Hamburg (DE)

(73) Assignee: Henkel AG & Co. KGaA, Duesseldorf (DE)

(21) Appl. No.: 17/786,448

(22) PCT Filed: Oct. 16, 2020
(86) PCT No.: PCT/EP2020/079171

§ 371 Date: Jun. 16, 2022

(30) Foreign Application Priority Data
Dec. 16, 2019 (DE) 102019219705.1

(51) Int. Cl. A61K 8/04; A61Q 5/06; A61Q 5/12; A61K 8/37; A61K 8/25

(52) U.S. Cl. CPC A61K 8/044; A61Q 5/06; A61Q 5/12; A61K 8/37; A61K 8/25; A61K 2800/882; A61K 2800/436

(57) ABSTRACT

A pigment suspension is disclosed. The pigment suspension comprises: (a) at least one coloring compound selected from the group of pigments; and (b) a hydroxycarboxylic acid ester. A cosmetic agent is also disclosed. The cosmetic agent comprises the pigment suspension, and one or more organic C₁-C₆ alkoxysilanes and/or condensation products thereof. A multi-component packaging unit (kit-of-parts) for dyeing keratinous material is also disclosed, and comprises separately packaged first and second containers. The first container comprises an agent (a′), which comprises one or more organic C₁-C₆-alkoxysilanes and/or condensation products thereof. The second container comprises an agent (a″), which comprises the pigment suspension.

### CROSS-REFERENCE TO RELATED APPLICATION

This application is a U.S. National-Stage entry under 35 U.S.C. § 371 based on International Application No. PCT/EP2020/079171, filed Oct. 16, 2020, which was published under PCT Article 21(2) and which claims priority to German Application No. 102019219705.1, filed Dec. 16, 2019, which are all hereby incorporated in their entirety by reference.

### TECHNICAL FIELD

The subject of the present application is a pigment suspension comprising a pigment and a carrier medium. Another article is a cosmetic agent prepared using the pigment suspension and an organosilicon compound.

Pigments, especially metal-comprising pigments, are frequently used in coatings, paints, printing inks, powder coatings, cosmetics or plastics for coloration. Paints, varnishes, printing inks, cosmetics and powder coatings are liquid or powder coating materials that are applied to surfaces to obtain both improved or altered optical and physical properties.

### BACKGROUND

The change in shape and color of keratin fibers, especially hair, is a key area of modern cosmetics. To change the hair color, the expert knows various coloring systems depending on coloring requirements. Oxidation dyes are usually used for permanent, intensive dyeings with good fastness properties and good grey coverage. Such dyes usually contain oxidation dye precursors, so-called developer components and coupler components, which form the actual dyes with one another under the influence of oxidizing agents, such as hydrogen peroxide. Oxidation dyes are exemplified by very long-lasting dyeing results.

When direct dyes are used, ready-made dyes diffuse from the colorant into the hair fiber. Compared to oxidative hair dyeing, the dyeings obtained with direct dyes have a shorter shelf life and quicker wash ability. Dyes with direct dyes usually remain on the hair for a period of between 5 and 20 washes.

The use of color pigments is known for short-term color changes on the hair and/or skin. Color pigments are understood to be insoluble, coloring substances. These are present undissolved in the dye formulation in the form of small particles and are only deposited from the outside on the hair fibers and/or the skin surface. Therefore, they can usually be removed without residue by a few washes with surfactant-comprising cleaning agents. Various products of this type are available on the market under the name hair mascara.

Metallic luster pigments or metallic effect pigments are widely used in many fields of technology. They are used, for example, to color coatings, printing inks, inks, plastics, glasses, ceramic products and preparations for decorative cosmetics such as nail polish. They are exemplified by their attractive angle-dependent color impression (goniochromism) and their metallic-looking luster.

Hair with a metallic finish or metallic highlights are in trend. The metallic tone makes the hair look thicker and shinier.

For applications in the cosmetic field, for example in the color modification of keratinic fibers with pigments, it is important that the pigments are provided to the user in a storage-stable and dosage-capable form. This can be done in the form of a storage-stable pigment suspension.

### BRIEF SUMMARY

A pigment suspension is provided. The pigment suspension comprises: (a) at least one coloring compound selected from the group of pigments; and (b) a hydroxycarboxylic acid ester.

A cosmetic agent is also provided. The cosmetic agent comprises (I) a pigment suspension, comprising: (a) at least one coloring compound selected from the group of pigments; and (b) a hydroxycarboxylic acid ester. The cosmetic agent also comprises (II) one or more organic C₁-C₆ alkoxysilanes and/or condensation products thereof.

A multi-component packaging unit (kit-of-parts) for dyeing keratinous material (e.g. human hair) is also provided. The multi-component packaging unit comprises separately packaged first and second containers. The first container comprises an agent (a′), which comprises one or more organic C₁-C₆-alkoxysilanes and/or condensation products thereof. The second container comprises an agent (a″), which comprises a pigment suspension comprising: (a) at least one coloring compound selected from the group of pigments; and (b) a hydroxycarboxylic acid ester.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Ground pigment powders and water are usually used to produce inorganic pigment suspensions. If necessary, organic or inorganic dispersing aids must be added in small quantities.

Some pigments, for example so-called metal effect pigments, have only limited stability in an aqueous agent. For example, aluminum-based metal effect pigments decompose comparatively rapidly in water to form hydrogen and aluminum hydroxide. To prevent this, the metallic pigment surfaces are conventionally protected by phosphating, chromating, silanizing or other coatings, for example of synthetic resin.

Such, coated metal effect pigments are more expensive than non-coated metal effect pigments.

It is the task of the present disclosure to provide pigment suspensions that can be prepared easily and inexpensively and are stable in storage. In particular, the pigments in the pigment suspension should be stable against decomposition and/or corrosion.

Pigment suspensions comprising a hydroxycarboxylic acid ester as a carrier medium have been shown to meet these requirements.

Accordingly, a first subject matter of the application is a comprising a) at least one colorant compound selected from the group of pigments and b) a hydroxycarboxylic acid ester.

As the first ingredient essential to the present disclosure, the pigment suspensions comprise at least one colorant compound from the group of pigments.

Pigments within the meaning of the present disclosure are coloring compounds which have a solubility in water at 25° C. of less than 0.5 g/L, preferably less than 0.1 g/L, even more preferably less than 0.05 g/L. Water solubility can be determined, for example, by the method described below: 0.5 g of the pigment are weighed in a beaker. A stir-fish is added. Then one liter of distilled water is added. This mixture is heated to 25° C. for one hour while stirring on a magnetic stirrer. If undissolved components of the pigment are still visible in the mixture after this period, the solubility of the pigment is below 0.5 g/L. If the pigment-water mixture cannot be assessed visually due to the high intensity of the finely dispersed pigment, the mixture is filtered. If a proportion of undissolved pigments remains on the filter paper, the solubility of the pigment is below 0.5 g/L.

Suitable pigments can be of inorganic and/or organic origin.

The at least one pigment preferably has a substrate platelet.

In principle, the substrate platelet can be made of any material that can be formed into a platelet shape. They can be of natural origin, but also synthetically produced. Materials from which the substrate platelets can be constructed include metals and metal alloys, metal oxides, preferably aluminum oxide, inorganic compounds and minerals such as mica and (semi-)precious stones, and plastics. Preferably, the substrate platelets are composed of a metal or alloy or a mica. The mica can be of natural or synthetic origin.

Accordingly, it may be preferred that the pigment comprises a substrate platelet, wherein the substrate platelet comprises a metal, metal alloy, natural mica or synthetic mica. In particular, the substrate plate is preferably made of a metal, a metal alloy, a natural mica or a synthetic mica.

Any metal suitable for pigments can be used. Such metals include iron and steel, as well as all air- and water-resistant (semi)metals such as platinum, tin, zinc, chromium, molybdenum and silicon, as well as their alloys such as aluminum bronzes and brass. Preferred metals are aluminum, copper, silver and gold.

In a particularly preferred embodiment, the pigment comprises a substrate platelet of a metal, wherein the metal is selected from the group of aluminum, copper, silver and gold, with substrate platelets of aluminum being particularly preferred.

Substrate platelets made of aluminum can be produced, among other things, by punching out of aluminum foil or according to common milling and atomization techniques. For example, aluminum flakes are available from the reverberation process, a wet milling process.

In another preferred embodiment, the pigment has a metal alloy substrate plate, wherein the metal alloy comprises brass.

In a particularly preferred embodiment, the pigment comprises a substrate platelet of a mica, wherein a substrate platelet of synthetic mica (INCI: Synthetic Fluorophlogopite) is particularly preferred.

A substrate platelet made of metal or a metal alloy preferably has an average thickness of at most about 150 nm, preferably less than about 50 nm, more preferably less than about 30 nm, particularly preferably at most about 25 nm, for example at most about 20 nm. The average thickness of the substrate platelets is at least about 1 nm, preferably at least about 2.5 nm, particularly preferably at least about 5 nm, for example at least about 10 nm. Preferred ranges for substrate platelets thickness are from about 2.5 to about 50 nm, from about 5 to about 50 nm, from about 10 to about 50 nm; from about 2.5 to about 30 nm, from about 5 to about 30 nm, from about 10 to about 30 nm; from about 2.5 to about 25 nm, from about 5 to about 25 nm, from about 10 to about 25 nm, from about 2.5 to about 20 nm, from about 5 to about 20 nm, and from about 10 to about 20 nm. Preferably, each substrate plate has a thickness that is as uniform as possible.

A substrate platelet of mica preferably has an average thickness of from about 50 to about 1500 nm and more preferably from about 90 to about 1000 nm.

The size of the substrate platelet can be tailored to the specific application, for example the desired effect on a keratinous material. Typically, the substrate platelets made of metal or a metal alloy have an average largest diameter of about 2 to about 200 μm, especially about 5 to about 100 μm. Typically, the substrate platelets of a mica have an average largest diameter of about 1 to about 200 μm, particularly about 5 to about 100 μm, and even more preferably about 5 to about 25 μm.

In a preferred embodiment, the shape factor (aspect ratio), expressed by the ratio of the average size to the average thickness, is at least about 80, preferably at least about 200, more preferably at least about 500, particularly preferably more than about 750. The average size of the uncoated substrate platelets is the d50 value of the uncoated substrate platelets. Unless otherwise stated, the d50 value was determined using a Sympatec Helos device with quixel wet dispersion. To prepare the sample, the sample to be analyzed was pre-dispersed in isopropanol for 3 minutes.

The substrate plates can have different shapes. For example, lamellar or lenticular metal platelets or so-called vacuum metallized pigments (VMP) can be used as substrate platelets. Lamellar substrate platelets are exemplified by an irregularly structured edge and are also referred to as “cornflakes” due to their appearance. Lenticular substrate platelets have a regular round edge and are also referred to as “silver dollars” due to their appearance.

The metal or metal alloy substrate plates can be passivated, for example by anodizing (oxide layer) or chromating.

A coating can change the surface properties and/or optical properties of the pigment and increase the mechanical and chemical resistance of the pigments. For example, only the upper and/or lower side of the substrate platelets may be coated, with the side surfaces being recessed. Preferably, the entire surface of the optionally passivated substrate platelets, including the side surfaces, is covered by the layer. The substrate platelets are preferably completely encased by the coating.

The coating may include one or more layers. In a preferred embodiment, the coating has only layer A. In a likewise preferred embodiment, the coating has a total of at least two, preferably two or three, layers. It may be preferred to have the coating have two layers A and B, with layer B being different from layer A. Preferably, layer A is located between layer B and the surface of the substrate plate. In yet another preferred embodiment, the coating has three layers A, B and C. In this embodiment, layer A is located between layer B and the surface of the substrate platelets and layer C is located on top of layer B, which is different from the layer B below.

Suitable materials for layers A and, if necessary, B and C are all substances that can be permanently applied to the substrate platelets. The materials should preferably be applicable in film form. Preferably, the entire surface of the optionally passivated substrate platelets, including the side surfaces, is enveloped by layer A or by layers A and B or by layers A, B and C.

In particular, the layers may each comprise at least one metal oxide (hydrate).

It is preferred that the metal oxide (hydrate) is selected from the group of silicon (di)oxide, silicon oxide hydrate, aluminum oxide, aluminum oxide hydrate, boron oxide, germanium oxide, manganese oxide, magnesium oxide, iron oxide, cobalt oxide, chromium oxide, titanium dioxide, vanadium oxide, zirconium oxide, tin oxide, zinc oxide and mixtures thereof.

In the case of pigments with a substrate platelet made of metal or a metal alloy, layer A preferably comprises at least one low-refractive metal oxide and/or metal oxide hydrate. Low refractive index materials have a refractive index of 1.8 or less, preferably 1.6 or less.

Low refractive index metal oxide (hydrate) suitable for Layer A include, for example, silicon (di)oxide, silicon oxide hydrate, aluminum oxide, aluminum oxide hydrate, boron oxide, germanium oxide, manganese oxide, magnesium oxide, and mixtures thereof, with silicon dioxide being preferred. Layer A preferably has a thickness of from about 1 to about 100 nm, particularly preferably from about 5 to about 50 nm, especially preferably from about 5 to about 20 nm.

Layer B, if present, is distinct from layer A and, in the case of pigments having a substrate platelet of metal or metal alloy, may comprise at least one highly refractive metal oxide (hydrate). Highly refractive materials have a refractive index of at least about 1.9, preferably at least about 2.0, and more preferably at least about 2.4. Preferably, layer B comprises at least about 95 wt. %, more preferably at least about 99 wt. %, of high refractive index metal oxide(s).

If the layer B comprises a (highly refractive) metal oxide, it preferably has a thickness of at least about 50 nm. Preferably, the thickness of layer B is no more than about 400 nm, more preferably no more than about 300 nm.

Highly refractive metal oxides suitable for layer B are, for example, selectively light-absorbing (i.e., colored) metal oxides, such as iron(III) oxide (α- and γ-Fe₂O₃, red), cobalt(II) oxide (blue), chromium(III) oxide (green), titanium(III) oxide (blue, usually present in admixture with titanium oxynitrides and titanium nitrides), and vanadium(V) oxide (orange), as well as mixtures thereof. Colorless high-index oxides such as titanium dioxide and/or zirconium oxide are also suitable.

Layer B can comprise a selectively absorbing dye in addition to a highly refractive metal oxide, preferably from about 0.001 to about 5 wt. %, particularly preferably from about 0.01 to about 1 wt. %, in each case based on the total amount of layer B. Suitable dyes are organic and inorganic dyes that can be stably incorporated into a metal oxide coating. Dyes in the sense of the present disclosure have a solubility in water (760 mmHg) at 25° C. of more than 0.5 g/L and are therefore not to be regarded as pigments.

As an alternative to a metal oxide, layer B in the case of pigments with a substrate platelet made of metal or a metal alloy may comprise a metal particle support layer with metal particles deposited on the surface of the metal particle support layer. In a preferred embodiment, the metal particles directly cover a portion of the metal particle carrier layer. In this embodiment, the effect pigment has areas in which there are no metal particles, i.e., areas which are not covered with the metal particles.

The metal particle carrier layer comprises a metal layer and/or a metal oxide layer.

If the metal particle carrier layer comprises a metal layer and a metal oxide layer, the arrangement of these layers is not limited.

It is preferred that the metal particle support layer at least comprises a metal layer. It is further preferred that the metal layer comprises an element selected from tin (Sn), palladium (Pd), platinum (Pt) and gold (Au).

The metal layer can be formed, for example, by adding alkali to a metal salt solution comprising the metal.

If the metal particle carrier layer comprises a metal oxide layer, this preferably does not comprise silicon dioxide. The metal oxide layer preferably comprises an oxide of at least one element selected from the group of Mg (magnesium), Sn (tin), Zn (zinc), Co (cobalt), Ni (nickel), Fe (iron), Zr (zirconium), Ti (titanium) and Ce (cerium). Particularly preferably, the metal particle support layer iii) in the form of a metal oxide layer comprises a metal oxide of Sn, Zn, Ti and Ce.

The metal particle support layer in the form of a metal oxide layer can be produced, for example, by hydrolysis of an alkoxide of a metal forming the metal of the metal oxide in a sol-gel process.

The thickness of the metal layer is preferably not more than 30 nm.

The metal particles may comprise at least one element selected from the group of aluminum (Al), titanium (Ti), chromium (Cr), iron (Fe), cobalt (Co), nickel (Ni), copper (Cu), zinc (Zn), ruthenium (Ru), rhodium (Rh), palladium (Pd), silver (Ag), tin (Sn), platinum (Pt), gold (Au), and alloys thereof. It is particularly preferred that the metal particles comprise at least one element selected from copper (Cu), nickel (Ni) and silver (Ag).

The average particle diameter of the metal particles is preferably not more than about 50 nm, more preferably not more than about 30 nm. The distance between the metal particles is preferably not more than about 10 m.

Suitable methods for forming the metal particles include vacuum evaporation, sputtering, chemical vapor deposition (CVD), electroless plating, or the like. Of these processes, electroless plating is particularly preferred.

According to a preferred embodiment, the pigments with a substrate platelet made of metal or a metal alloy have a further layer C comprising a metal oxide (hydrate), which is different from the layer B underneath. Suitable metal oxides include silicon (di)oxide, silicon oxide hydrate, aluminum oxide, aluminum oxide hydrate, zinc oxide, tin oxide, titanium dioxide, zirconium oxide, iron (III) oxide, and chromium (III) oxide. Silicon dioxide is preferred.

In the case of pigments with a substrate platelet made of metal or a metal alloy, layer C preferably has a thickness of from about 10 to about 500 nm, particularly preferably from about 50 to about 300 nm.

Suitable pigments based on a substrate platelet made of a metal or metal alloy are, for example, the pigments Alegrace® Marvelous, Alegrace© Gorgeous or Alegrace® Aurous from Schlenk Metallic Pigments.

In the case of pigments with a substrate platelet of mica, in particular synthetic mica, layer A comprises a metal oxide (hydrate) selected from the group of titanium dioxide (TiO₂), iron oxide (Fe₂O₃ and/or Fe₃O₄) and mixtures thereof. In a very preferred embodiment, layer A comprises titanium dioxide (TiO₂) and/or iron oxide (Fe₂O₃). In a highly preferred embodiment, layer A comprises titanium dioxide (TiO₂).

Layer B, if present, is also different from the first metal oxide (hydrate) layer in the case of pigments with a substrate platelet of mica.

Metal oxide (hydrate)s suitable for layer B are tin oxide (SnO₂), silicon oxide (SiO₂), aluminum oxide (Al₂O₃) and/or iron oxide (Fe₂O₃ and/or Fe₃O₄). Accordingly, it is preferred that layer B comprises a metal oxide (hydrate) selected from the group of tin oxide (SnO₂), silicon oxide (SiO₂), aluminum oxide (Al₂O₃), iron oxide (Fe₂O₃ and/or Fe₃O₄), and mixtures thereof. It is particularly preferred that layer B comprises tin oxide (SnO₂) in the case of pigments with a substrate platelet of mica, preferably synthetic mica.

Layer B may further comprise a selectively absorbing dye or pigment. Suitable dyes and/or pigments include, for example, carmine, ferric hexacyanidoferrate(II/III), and chromium oxide green (Cr₂O₃).

The pigments with a substrate platelet of mica may have another layer C, which acts as a protective layer and comprises a metal oxide (hydrate) or a polymer, for example a synthetic resin. Suitable metal oxide (hydrates)s includes silicon (di)oxide, silicon oxide hydrate, aluminum oxide, aluminum oxide hydrate, zinc oxide, tin oxide, titanium dioxide, zirconium oxide, iron (III) oxide, and chromium (III) oxide. Silicon dioxide is preferred.

It is particularly preferred that a pigment with a substrate platelet of synthetic mica (INCI: Synthetic Fluorophlogopite) has a layer A comprising titanium dioxide (TiO₂).

It is also preferred that a pigment with a substrate platelet of synthetic mica (INCI: Synthetic Fluorophlogopite) has a layer A comprising iron(III) oxide (Fe₂O₃).

It is also preferred that a pigment with a substrate platelet of synthetic mica (INCI: Synthetic Fluorophlogopite) has a layer A comprising titanium dioxide (TiO₂) and iron(III) oxide (Fe₂O₃), and a layer B comprising tin dioxide (SnO₂).

It is highly preferred that a pigment with a substrate platelet of synthetic mica (INCI: Synthetic Fluorophlogopite) has a layer A comprising titanium dioxide (TiO₂) and a layer B comprising tin dioxide (SnO₂).

A preferred pigment with a substrate platelet of synthetic mica is available, for example, under the name Timiron SynWhite Satin from Merck.

It is also particularly preferred that the pigment comprises at least one pigment of organic origin.

The organic pigments are correspondingly insoluble organic dyes or colorants which may be selected, for example, from the group of nitroso, nitro-azo, xanthene, anthraquinone, isoindolinone, isoindoline, quinacridone, perinone, perylene, diketopyrrolopyorrole, indigo, thioindido, dioxazine and/or triarylmethane compounds.

Examples of particularly suitable organic pigments are carmine, quinacridone, phthalocyanine, sorghum, blue pigments with the Color Index numbers CI 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

Highly preferred pigment suspensions are correspondingly wherein the pigment comprises at least one organic pigment selected from the group of carmine, quinacridone, phthalocyanine, Sorgho, blue pigments with the color index numbers CI 42090, CI 69800, CI 69825, CI 73000, CI 74100, CI 74160, yellow pigments with the color index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, CI 75470 and mixtures thereof.

The amount of pigment in the pigment suspension depends in particular on the type of pigment(s) and its intended use. Preferably, the amount of pigment is between about 1 and about 90 wt. %, more preferably between about 5 and about 80 wt. % and most preferably between about 10 and about 70 wt. %, in each case based on the total weight of the pigment suspension.

In addition to the above-mentioned, particularly preferred pigments with substrate platelets made of metal, a metal alloy or mica, as well as the organic pigments, further color-imparting compounds can be included in the pigment suspension. The further colorant compounds may comprise, for example, further inorganic pigments and/or direct-acting dyes.

As a second ingredient essential to the present disclosure, the pigment suspension comprises a hydroxycarboxylic acid ester.

Hydroxycarboxylic acids are carboxylic acids that have both at least one carboxy group and one or more hydroxy group(s). Hydroxycarboxylic acids represent widespread organic substances in nature.

Suitable hydroxycarboxylic acids include, for example, α-hydroxycarboxylic acids and/or β-hydroxycarboxylic acids. Particularly suitable hydroxycarboxylic acids are selected from the group of citric acid, malic acid, tartaric acid, lactic acid, gluconic acid, glycolic acid, tartronic acid, mandelic acid, salicylic acid, glyceric acid and mixtures thereof. Particularly preferred is the hydroxycarboxylic acid selected from the group of citric acid, malic acid, tartaric acid, lactic acid and mixtures thereof. The hydroxycarboxylic acid citric acid is particularly preferred.

Accordingly, in a preferred embodiment, pigment suspension is wherein it comprises at least one hydroxycarboxylic acid ester (a2) selected from the group of citric acid esters, malic acid esters, tartaric acid esters, lactic acid esters, gluconic acid esters, glycolic acid esters, tartronic acid esters, mandelic acid esters, salicylic acid esters, glyceric acid esters and mixtures thereof.

In a more preferred embodiment, the pigment suspension is exemplified by comprising at least one hydroxycarboxylic acid ester selected from the group of citric acid esters, malic acid esters, tartaric acid esters, lactic acid esters, and mixtures thereof.

In a highly preferred embodiment, the pigment suspension is wherein it comprises at least one hydroxycarboxylic acid ester selected from the group of citric acid esters.

Hydroxycarboxylic acid esters are the esters of hydroxycarboxylic acids with aliphatic alcohols. The aliphatic alcohols are linear or branched, saturated or unsaturated alcohols with 1 to 22 carbon atoms and 0, 1, 2 or 3 double bonds. Typical representatives are, for example, methanol, ethanol, n-propanol, isopropanol, n-butanol, sec.-butanol, tert, butanol, n-pentanol, capric alcohol, caprylic alcohol, 2-ethylhexanol, capric alcohol, myristyl alcohol, lauryl alcohol, cetyl alcohol, palmitoleyl alcohol, stearyl alcohol, oleyl alcohol, elaidyl alcohol, petroselyl alcohol, linolyl alcohol, linolenyl alcohol, behenyl alcohol or erucyl alcohol, Preferably, the aliphatic alcohols are linear or branched, saturated alcohols having 1 to 6 carbon atoms, which are preferably selected from the group of ethanol, n-propanol, isopropanol, n-butanol, sec-butanol, tert-butanol and mixtures thereof. Very preferably, the aliphatic alcohol is ethanol.

Accordingly, in a preferred embodiment, the pigment suspension is exemplified by comprising at least one hydroxycarboxylic acid ester selected from the group of citric acid triethyl ester (triethyl citrate), malic acid diethyl ester (diethyl malate), tartaric acid diethyl ester (diethyl tartrate), lactic acid ethyl ester (ethyl lactate), and mixtures thereof.

In a preferred embodiment, the pigment suspension is wherein it comprises at least one hydroxycarboxylic acid ester comprising citric acid triethyl ester.

Preferably, the amount of hydroxycarboxylic acid ester is between about 10 and about 99 wt. %, more preferably between about 20 and about 95 wt. % and most preferably between about 30 and about 90 wt. %, in each case based on the total weight of the pigment suspension.

It has been shown that the pigments, especially those with substrate platelets of metal, metal alloy, natural mica or synthetic mica, are protected from decomposition and can be accurately metered. Pigments with substrate platelets made of metal or a metal alloy are protected against corrosion.

The present disclosure also relates to a cosmetic composition. This was prepared by combining a pigment suspension according to the present disclosure with one or more organic C₁-C₆ alkoxysilanes and/or their condensation products. In this way, cosmetic agents can be provided that contain all the desired components beneficial to the cosmetic agent, with the pigments protected from decomposition and the C₁-C₆ alkoxysilanes protected from hydrolysis.

Such cosmetic agents can be used, for example, in processes for coloring keratinous material, especially human hair.

The cosmetic agent is wherein it comprises one or more organic C₁-C₆ alkoxysilanes and/or their condensation products.

The organic C₁-C₆ alkoxysilane(s) are organic, non-polymeric silicon compounds, preferably selected from the group of silanes having one, two or three silicon atoms

Organic silicon compounds, alternatively called organosilicon compounds, are compounds which either have a direct silicon-carbon bond (Si—C) or in which the carbon is bonded to the silicon atom via an oxygen, nitrogen or sulfur atom. The organic silicon compounds of the present disclosure are preferably compounds comprising one to three silicon atoms. Organic silicon compounds preferably contain one or two silicon atoms.

According to IUPAC rules, the term silane stands for a group of chemical compounds based on a silicon basic framework and hydrogen. In organic silanes, the hydrogen atoms are completely or partially replaced by organic groups such as (substituted) alkyl groups and/or alkoxy groups.

A characteristic feature of the C₁-C₆ alkoxysilanes as contemplated herein is that at least one C₁-C₆ alkoxy group is directly bonded to a silicon atom. The C₁-C₆ alkoxysilanes as contemplated herein thus comprise at least one structural unit R′R″R′″Si—O—(C₁-C₆ alkyl) where the radicals R′, R″ and R′″ represent the three remaining bond valencies of the silicon atom.

The C₁-C₆ alkoxy group or groups bonded to the silicon atom are very reactive and are hydrolyzed at high rates in the presence of water, the reaction rate depending, among other things, on the number of hydrolysable groups per molecule. If the hydrolysable C₁-C₆ alkoxy group is an ethoxy group, the organic silicon compound preferably comprises a structural unit R′R″R′″ Si—O—CH₂—CH₃. The radicals R′, R″ and R′″ again represent the three remaining free valences of the silicon atom.

Even the addition of lesser amounts of water leads first to hydrolysis and then to a condensation reaction between the organic alkoxysilanes. For this reason, both the organic alkoxysilanes and their condensation products may be present in the composition.

A condensation product is understood to be a product formed by the reaction of at least two organic C₁-C₆ alkoxysilanes with elimination of water and/or with elimination of a C₁-C₆ alkanol.

The condensation products can, for example, be dimers, or even trimers or oligomers, where in the condensation products are always in balance with the monomers.

Depending on the amount of water used or consumed in the hydrolysis, the equilibrium shifts from monomeric C₁-C₆ alkoxysilane to condensation product.

In a very particularly preferred embodiment, a cosmetic agent is wherein the cosmetic agent comprises one or more organic C₁-C₆ alkoxysilanes selected from silanes having one, two or three silicon atoms, the organic silicon compound further comprising one or more basic chemical functions.

This basic group can be, for example, an amino group, an alkylamino group or a dialkylamino group, which is preferably connected to a silicon atom via a linker. Preferably, the basic group is an amino group, a C₁-C₆ alkylamino group or a di(C₁-C₆)alkylamino group.

A very particularly preferred agent is wherein the cosmetic agent comprises one or more organic C₁-C₆ alkoxysilanes selected from the group of silanes having one, two or three silicon atoms, and wherein the C₁-C₆ alkoxysilanes further comprise one or more basic chemical functions.

Particularly satisfactory results were obtained when C₁-C₆ alkoxysilanes of the formula (S-I) and/or (S-II) and/or (S-IV) were used in the cosmetic agent. Since, as previously described, hydrolysis/condensation already starts at traces of moisture, the condensation products of the C₁-C₆ alkoxysilanes of formula (S-I) and/or (S-II) and/or (S-IV) are also included in this embodiment.

In another very particularly preferred embodiment, the cosmetic agent is wherein it comprises one or more organic C₁-C₆ alkoxysilanes of the formula (S-I) and/or (S-II),

R₁R₂N-L-Si(OR₃)ₐ(R₄)_{b} (S-I)

where
R₁, R₂ independently represent a hydrogen atom or a C₁-C₆ alkyl group,
L is a linear or branched divalent C₁-C₂₀ alkylene group,
R₃, R₄ independently of one another represent a C₁-C₆ alkyl group,
a, stands for an integer from 1 to 3, and
b stands for the integer 3-a, and

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (S-II),

where
R5, R5′, R5″, R6, R6′ and R6″ independently represent a C₁-C₆ alkyl group,
A, A′, A″, A′″ and A″″ independently represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (S-III),

(A″″)-Si(R₆″)_{d}″(OR₅″)_{c}″ (S-III),
c, stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
provided that at least one of e, f, g and h radicals are different from 0, and/or their condensation products.

The substituents R₁, R₂, R₃, R₄, R₅, R₅′, R₅″, R₆, R₆′, R₆″, R₇, R₈, L, A, A′, A″, A′″ and A″″ in the compounds of formula (S-I) and (S-II) are explained below as examples:

Examples of a C₁-C₆ alkyl group are the groups methyl, ethyl, propyl, isopropyl, n-butyl, s-butyl and t-butyl, n-pentyl and n-hexyl. Propyl, ethyl and methyl are preferred alkyl radicals. Examples of a C₂-C₆ alkenyl group are vinyl, allyl, but-2-enyl, but-3-enyl and isobutenyl, preferred C₂-C₆ alkenyl radicals are vinyl and allyl. Preferred examples of a hydroxy C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly preferred. Examples of an amino C₁-C₆ alkyl group are the aminomethyl group, the 2-aminoethyl group, the 3-aminopropyl group. The 2-aminoethyl group is particularly preferred. Examples of a linear bivalent C₁-C₂₀ alkylene group include the methylene group (—CH₂—), the ethylene group (—CH₂—CH₂—), the propylene group (—CH₂—CH₂—CH₂—), and the butylene group (—CH₂—CH₂—CH₂—CH₂—). The propylene group (—CH₂—CH₂—CH₂—) is particularly preferred. From a chain length of 3 C atoms, bivalent alkylene groups can also be branched. Examples of branched divalent, bivalent C₃-C₂₀ alkylene groups are (—CH₂—CH(CH₃)—) and (—CH₂—CH(CH₃)—CH₂—).

In the organic silicon compounds of the formula (S-I)

R₁R₂N-L-Si(OR₃)ₐ(R₄)_{b} (S-I),

the radicals R₁ and R₂ independently of one another represent a hydrogen atom or a C₁-C₆ alkyl group. Very preferably, radicals R₁ and R₂ both represent a hydrogen atom.

In the middle part of the organic silicon compound is the structural unit or the linker -L- which stands for a linear or branched, divalent C₁-C₂₀ alkylene group. The divalent C₁-C₂₀ alkylene group may alternatively be referred to as a divalent or divalent C₁-C₂₀ alkylene group, by which is meant that each -L grouping may form- two bonds.

Preferably -L- stands for a linear, bivalent C₁-C₂₀ alkylene group. Further preferably -L- stands for a linear bivalent C₁-C₆ alkylene group. Particularly preferred -L stands for a methylene group (CH₂—), an ethylene group (—CH₂—CH₂—), propylene group (—CH₂—CH₂—CH₂—) or butylene (—CH₂—CH₂—CH₂—CH₂—). In some embodiments, L stands for a propylene group (—CH₂—CH₂—CH₂—).

The alkoxysilanes of the formula (S-I)

R₁R₂N-L-Si(OR₃)ₐ(R₄)_{b} (S-I),

one end of each carries the silicon-comprising group —Si(OR₃)ₐ(R₄)_{b}.

In the terminal structural unit —Si(OR₃)ₐ(R₄)_{b} radicals R₃ and R₄ independently represent a C₁-C₆ alkyl group, and particularly preferably R₃ and R₄ independently represent a methyl group or an ethyl group.

Here a stands for an integer from 1 to 3, and b stands for the integer 3-a. If a stands for the number 3, then b is equal to 0. If a stands for the number 2, then b is equal to 1. If a stands for the number 1, then b is equal to 2.

Cosmetic agents with particularly good coloring properties for keratinous materials could be prepared if the agent comprises at least one organic C₁-C₆ alkoxysilane of the formula (S-I) in which the radicals R₃, R₄ independently of one another represent a methyl group or an ethyl group.

Furthermore, colorations with the best wash fastnesses could be obtained if the cosmetic agent comprises at least one organic C₁-C₆ alkoxysilane of the formula (S-I) in which the radical a represents the number 3. In this case the radical b stands for the number 0.

In another preferred embodiment, a cosmeceutical agent is wherein it comprises one or more organic C₁-C₆ alkoxysilanes of formula (S-I),

where
R₃, R₄ independently of one another represent a methyl group or an ethyl group and
a stands for the number 3 and
b stands for the number 0.

In another preferred embodiment, a cosmetic agent is wherein the agent comprises at least one or more organic C₁-C₆ alkoxysilanes of the formula (S-I),

R₁R₂N-L-Si(OR₃)ₐ(R₄)_{b} (S-I),

where
R₁, R₂ both represent a hydrogen atom, and
L represents a linear, bivalent C₁-C₆-alkylene group, preferably a propylene group (—CH₂—CH₂—CH₂—) or an ethylene group (—CH₂—CH₂—),
R₃ represents an ethyl group or a methyl group,
R₄ represents a methyl group or an ethyl group,
a stands for the number 3 and
b stands for the number 0.

Particularly well suited organic silicon compounds of formula (I) are
(3-Aminopropyl)triethoxysilane

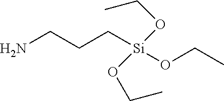

(3-Aminopropyl)trimethoxysilane

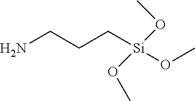

(2-Aminoethyl)triethoxysilane

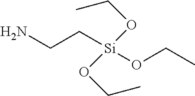

(2-Aminoethyl)trimethoxysilane

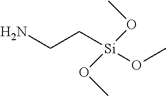

(3-Dimethylaminopropyl)triethoxysilane

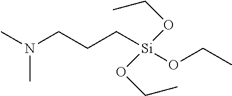 [SMILES: CCO[Si](CCCN(C)C)(OCC)OCC]

(3-Dimethylaminopropyl)trimethoxysilane

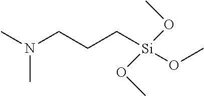

(2-Dimethylaminoethyl)triethoxysilane.

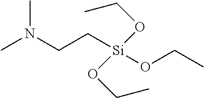

(2-Dimethylaminoethyl)trimethoxysilane and/or

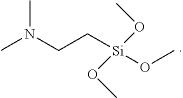

In a further preferred embodiment, the cosmetic agent is wherein it comprises at least one organic C₁-C₆ alkoxysilane of the formula (S-I) selected from the group of
(3-Aminopropyl)triethoxysilane
(3-Aminopropyl)trimethoxysilane
(2-Aminoethyl)triethoxysilane
(2-Aminoethyl)trimethoxysilane
(3-Dimethylaminopropyl)triethoxysilane
(3-Dimethylaminopropyl)trimethoxysilane
(2-Dimethylaminoethyl)triethoxysilane,
(2-Dimethylaminoethyl)trimethoxysilane
   and/or their condensation products.

The organic silicon compound of formula (I) is commercially available. (3-aminopropyl)trimethoxysilane, for example, can be purchased from Sigma-Aldrich. Also (3-aminopropyl)triethoxysilane is commercially available from Sigma-Aldrich.

In another embodiment, the cosmetic agent may also comprise one or more organic C₁-C₆ alkoxysilanes of formula (S-II),

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c}′ (S-II).

The alkoxysilanes of the formula (S-II) carry at both ends the silicon-comprising groups (R₅O)_{c}(R₆)_{d}Si— and —Si(R₆′)_{d′}(OR₅′)_{c′}.

In the central part of the molecule of formula (S-II) there are the groups -(A)ₑ- and —[NR₇-(A′)]_{f}- and —[O-(A″)]_{g}- and —[NR₈-(A′″)]ₕ-. Here, each of the radicals e, f, g and h can independently of one another stand for the number 0 or 1, with the proviso that at least one of the radicals e, f, g and h is different from 0. In other words, a preferred alkoxysilane of formula (II) comprises at least one moiety selected from the group of -(A)- and —[NR₇-(A′)]- and —[O-(A″)]- and —[NR₈-(A′″)]-.

In the two terminal structural units (R₅O)_{c}(R₆)_{d}Si— and —Si(R₆′)_{d′}(OR₅′)_{c}′, the radicals R5, R5′, R5″ independently represent a C₁-C₆ alkyl group. The radicals R6, R6′ and R6″ independently represent a C₁-C₆ alkyl group.

Here c stands for an integer from 1 to 3, and d stands for the integer 3-c. If c stands for the number 3, then d is equal to 0. If c stands for the number 2, then d is equal to 1. If c stands for the number 1, then d is equal to 2.

Analogously c′ stands for a whole number from 1 to 3, and d′ stands for the whole number 3-c′. If c′ stands for the number 3, then d′ is 0. If c′ stands for the number 2, then d′ is 1. If c′ stands for the number 1, then d′ is 2.

Dyeings with the best wash fastness values could be obtained if the residues c and c′ both stand for the number 3. In this case d and d′ both stand for the number 0.

In another preferred embodiment, a cosmetic agent is wherein it comprises one or more organic C₁-C₆ alkoxysilanes of the formula (S-II),

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c}′ (S-II),

where
R5 and R5′ independently represent a methyl group or an ethyl group,
c and c′ both stand for the number 3 and
d and d′ both stand for the number 0.

When c and c′ are both 3 and d and d′ are both 0, the organic silicon compounds as contemplated herein correspond to the formula (S-IIa)

(R₅O)₃Si-(A)ₑ-[NR₇-(A′)]_{f}-[O-(A″)]_{g}-[NR₈-(A′″)]ₕ-Si(OR₅′)₃ (S-IIa).

The radicals e, f, g and h can independently stand for the number 0 or 1, whereby at least one radical from e, f, g and h is different from zero. The abbreviations e, f, g and h thus define which of the groupings -(A)ₑ- and —[NR₇-(A′)]_{f}- and —[O-(A″)]_{g}- and —[NR₈-(A′″)]ₕ- are in the middle part of the organic silicon compound of formula (II).

In this context, the presence of certain groupings has proven to be particularly advantageous in terms of achieving washfast dyeing results. Particularly satisfactory results could be obtained if at least two of the residues e, f, g and h stand for the number 1. Especially preferred e and f both stand for the number 1. Furthermore, g and h both stand for the number 0.

When e and f are both 1 and g and h are both 0, the organic silicon compounds as contemplated herein are represented by the formula (S-IIb)

(R₅O)_{c}(R₆)_{d}Si-(A)-[NR₇-(A′)]-Si(R₆′)_{d′}(OR₅′)_{c′} (S-IIb).

The radicals A, A′, A″, A′″ and A″″ independently represent a linear or divalent, bivalent C₁-C₂₀ alkylene group. Preferably the radicals A, A′, A″, A′″ and A″″ independently of one another represent a linear, bivalent C₁-C₂₀ alkylene group. Further preferably the radicals A, A′, A″, A′″ and A″″ independently represent a linear bivalent C₁-C₆ alkylene group.

The divalent C₁-C₂₀ alkylene group may alternatively be referred to as a divalent or divalent C₁-C₂₀ alkylene group, by which is meant that each grouping A, A′, A″, A′″ and A″″ may form two bonds.

In particular, the radicals A, A′, A″, A′″ and A″″ independently of one another represent a methylene group (—CH₂—), an ethylene group (—CH₂—CH₂—), a propylene group (—CH₂—CH₂—CH₂—) or a butylene group (—CH₂—CH₂—CH₂—CH₂—). Very preferably, the radicals A, A′, A″, A′″ and A″″ represent a propylene group (—CH₂—CH₂—CH₂—).

If the radical f represents the number 1, then the organic silicon compound of formula (II) as contemplated herein comprises a structural grouping —[NR₇-(A′)]-. If the radical h represents the number 1, then the organic silicon compound of formula (II) as contemplated herein comprises a structural grouping —[NR₈-(A′″)]-.

Wherein R₇ and R₈ independently represent a hydrogen atom, a C₁-C₆ alkyl group, a hydroxy-C₁-C₆ alkyl group, a C₂-C₆ alkenyl group, an amino-C₁-C₆ alkyl group or a group of the formula (S-III)

-(A″″)—Si(R₆″)_{d″}(OR₅″)_{c}″ (S-III).

Very preferably the radicals R₇ and R₈ independently of one another represent a hydrogen atom, a methyl group, a 2-hydroxyethyl group, a 2-alkenyl group, a 2-aminoethyl group or a grouping of the formula (S-III).

When the radical f represents the number 1 and the radical h represents the number 0, the organic silicon compound as contemplated herein comprises the grouping [NR₇-(A′)] but not the grouping —[NR₈-(A″)]. If the radical R₇ now stands for a grouping of the formula (III), the organic silicon compound comprises 3 reactive silane groups.

In another preferred embodiment, a cosmetic agent is wherein the cosmetic agent comprises one or more organic C₁-C₆ alkoxysilanes of the formula (S-II)

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

where
e and f both stand for the number 1,
g and h both stand for the number 0,
A and A′ independently represent a linear, divalent C₁-C₆ alkylene group and
R7 represents a hydrogen atom, a methyl group, a 2-hydroxyethyl group, a 2-alkenyl group, a 2-aminoethyl group or a group of formula (S-III).

In a further preferred embodiment, the cosmetic agent is wherein it comprises one or more organic C₁-C₆ alkoxysilanes of the formula (S-II), wherein
e and f both stand for the number 1,
g and h both stand for the number 0,
A and A′ independently of one another represent a methylene group (—CH₂—), an ethylene group (—CH₂—CH₂—) or a propylene group (—CH₂—CH₂—CH₂), and
R7 represents a hydrogen atom, a methyl group, a 2-hydroxyethyl group, a 2-alkenyl group, a 2-aminoethyl group or a group of formula (S-III).

Well-suited organic silicon compounds of the formula (S-II) are
3-(Trimethoxysilyl)-N-[3-(trimethoxysilyl)propyl]-1-propanamine

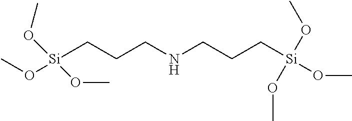

3-(Triethoxysilyl)-N-[3-(triethoxysilyl) propyl]-1-propanamine

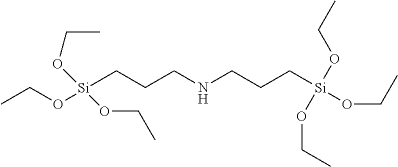 [SMILES: CCO[Si](CCCNCCC[Si](OCC)(OCC)OCC)(OCC)OCC]

N-methyl-3-(trimethoxysilyl)-N-[3-(trimethoxysilyl)propyl]-1-propanamine

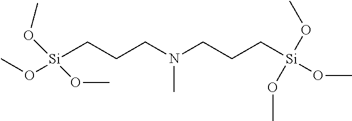

N-Methyl-3-(triethoxysilyl)-N-[3-(triethoxysilyl)propyl]-1-propanamine

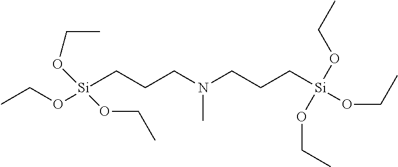 [SMILES: CCO[Si](CCCN(C)CCC[Si](OCC)(OCC)OCC)(OCC)OCC]

2-[Bis[3-(trimethoxysilyl)propyl]amino]-ethanol

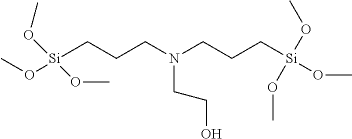

2-[Bis[3-(triethoxysilyl)propyl]amino]ethanol

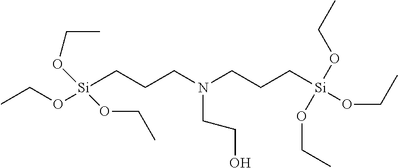 [SMILES: CCO[Si](CCCN(CCO)CCC[Si](OCC)(OCC)OCC)(OCC)OCC]

3-(Trimethoxysilyl)-N,N-bis[3-(trimethoxysilyl)propyl]-1-propanamine

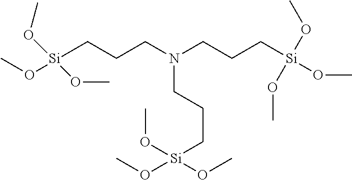

3-(Triethoxysilyl)-N,N-bis[3-(triethoxysilyl)propyl]-1-propanamine

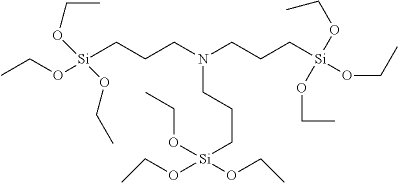 [SMILES: CCO[Si](CCCN(CCC[Si](OCC)(OCC)OCC)CCC[Si](OCC)(OCC)OCC)(OCC)OCC]

N1,N1-Bis[3-(trimethoxysilyl)propyl]-1,2-ethanediamine,

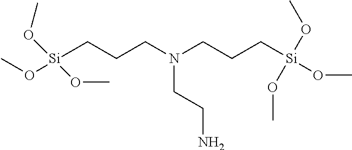

N1,N1-Bis[3-(triethoxysilyl)propyl]-1,2-ethanediamine,

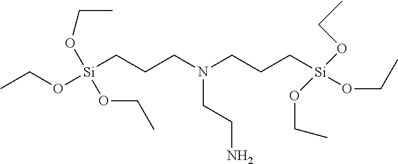 [SMILES: CCO[Si](CCCN(CCN)CCC[Si](OCC)(OCC)OCC)(OCC)OCC]

N,N-Bis[3-(trimethoxysilyl)propyl]-2-propene-1-amine

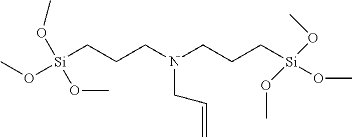

N,N-Bis[3-(triethoxysilyl)propyl]-2-propene-1-amine

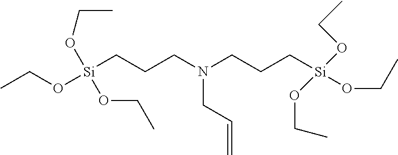 [SMILES: C=CCN(CCC[Si](OCC)(OCC)OCC)CCC[Si](OCC)(OCC)OCC]

The organic silicon compounds of formula (S-II) are commercially available. Bis(trimethoxysilylpropyl)amines with the CAS number 82985-35-1 can be purchased from Sigma-Aldrich.

Bis[3-(triethoxysilyl)propyl]amines with the CAS number 13497-18-2 can be purchased from Sigma-Aldrich, for example.

N-methyl-3-(trimethoxysilyl)-N-[3-(trimethoxysilyl)propyl]-1-propanamine is alternatively referred to as Bis(3-trimethoxysilylpropyl)-N-methylamine and can be purchased commercially from Sigma-Aldrich or Fluorochem. 3-(triethoxysilyl)-N,N-bis[3-(triethoxysilyl)propyl]-1-propanamine with the CAS number 18784-74-2 can be purchased for example from Fluorochem or Sigma-Aldrich.

In another preferred embodiment, a cosmetic agent is wherein the cosmetic agent comprises one or more organic C₁-C₆ alkoxysilanes of formula (S-II) selected from the group of
3-(Trimethoxysilyl)-N-[3-(trimethoxysilyl)propyl]-1-propanamine
3-(Triethoxysilyl)-N-[3-(triethoxysilyl) propyl]-1-propanamine
N-Methyl-3-(trimethoxysilyl)-N-[3-(trimethoxysilyl)propyl]-1-propanamine
N-Methyl-3-(triethoxysilyl)-N-[3-(triethoxysilyl) propyl]-1-propanamine
2-[Bis[3-(trimethoxysilyl) propyl]amino]-ethanol
2-[Bis[3-(triethoxysilyl) propyl]amino]ethanol
3-(Trimethoxysilyl)-N,N-bis[3-(trimethoxysilyl) propyl]-1-propanamine
3-(Triethoxysilyl)-N,N-bis[3-(triethoxysilyl) propyl]-1-propanamine
N1,N1-Bis[3-(trimethoxysilyl) propyl]-1,2-ethanediamine,
N1,N1-Bis[3-(triethoxysilyl) propyl]-1,2-ethanediamine,
N,N-Bis[3-(trimethoxysilyl)propyl]-2-Propen-1-amine and/or
N,N-Bis[3-(triethoxysilyl)propyl]-2-propen-1-amine,
   and/or their condensation products.

In dyeing tests, it has also been found to be particularly advantageous if the cosmetic agent comprises at least one organic C₁-C₆ alkoxysilane of the formula (S-IV)

R₉Si(OR₁₀)ₖ(R₁₁)ₘ (S-IV).

The compounds of formula (S-IV) are organic silicon compounds selected from silanes having one, two or three silicon atoms, wherein the organic silicon compound comprises one or more hydrolysable groups per molecule.

The organic silicon compound(s) of formula (S-IV) may also be referred to as silanes of the alkyl-C₁-C₆ alkoxysilane type,

R₉Si(OR₁₀)ₖ(R₁₁)ₘ (S-IV),

where
R₉ represents a C₁-C₁₂ alkyl group,
R₁₀ represents a C₁-C₆ alkyl group,
R₁₁ represents a C₁-C₆ alkyl group
k is an integer from 1 to 3, and
m stands for the integer 3-k.

In a further embodiment, a particularly preferred cosmetic agent is wherein it comprises one or more organic C₁-C₆ alkoxysilanes of the formula (S-IV),

R₉Si(OR₁₀)ₖ(R₁₁)ₘ (S-IV),

where
R₉ represents a C₁-C₁₂ alkyl group,
R₁₀ represents a C₁-C₆ alkyl group,
R₁ represents a C₁-C₆ alkyl group
k is an integer from 1 to 3, and
m stands for the integer 3-k,

and/or their condensation products.

In the organic C₁-C₆ alkoxysilanes of formula (S-IV), the radical R₉ represents a C₁-C₁₂ alkyl group. This C₁-C₁₂ alkyl group is saturated and can be linear or branched. Preferably, R₉ represents a linear C₁-C₈ alkyl group. Preferably R₉ stands for a methyl group, an ethyl group, an n-propyl group, an n-butyl group, an n-pentyl group, an n-hexyl group, an n-octyl group or an n-dodecyl group. Particularly preferred, R₉ stands for a methyl group, an ethyl group or an n-octyl group.

In the alkoxysilanes of formula (S-IV), the radical R₁₀ represents a C₁-C₆ alkyl group. Highly preferred R₁₀ stands for a methyl group or an ethyl group.

In the alkoxysilanes of formula (S-IV), the radical R₁₁ represents a C₁-C₆ alkyl group. Highly preferred R₁₁ stands for a methyl group or an ethyl group.

Furthermore, k stands for a whole number from 1 to 3, and m stands for the whole number 3-k. If k stands for the number 3, then m is equal to 0. If k stands for the number 2, then m is equal to 1. If k stands for the number 1, then m is equal to 2.

Colorations with the best wash fastnesses could be obtained when the cosmetic agent comprises at least one organic C₁-C₆ alkoxysilane of formula (S-IV), in which the radical k represents the number 3. In this case the radical m stands for the number 0.

Particularly suitable organic silicon compounds of the formula (S-IV) are
Methyltrimethoxysilane

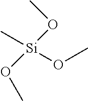

Methyltriethoxysilane

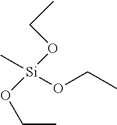

Ethyltrimethoxysilane

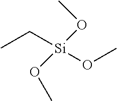

Ethyltriethoxysilane

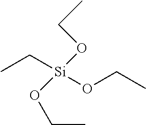

n-Propyltrimethoxysilane (also known as propyltrimethoxysilane)

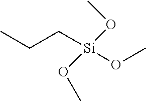

n-Propyltriethoxysilane (also known as propyltriethoxysilane)

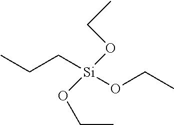

n-Hexyltrimethoxysilane (also known as hexyltrimethoxysilane)

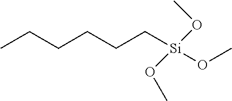

n-Hexyltriethoxysilane (also known as hexyltriethoxysilane)

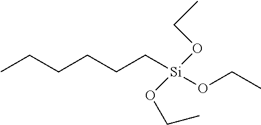 [SMILES: CCCCCC[Si](OCC)(OCC)OCC]

n-Octyltrimethoxysilane (also known as octyltrimethoxysilane)

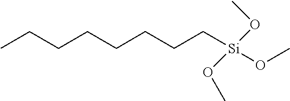

n-Octyltriethoxysilane (also known as octyltriethoxysilane)

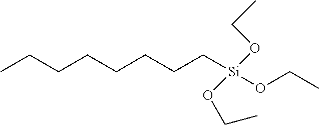 [SMILES: CCCCCCCC[Si](OCC)(OCC)OCC]

n-Dodecyltrimethoxysilane (also known as dodecyltrimethoxysilane) and/or

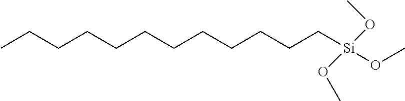 [SMILES: CCCCCCCCCCCC[Si](OC)(OC)OC]

n-Dodecyltriethoxysilanes (also known as dodecyltriethoxysilane)

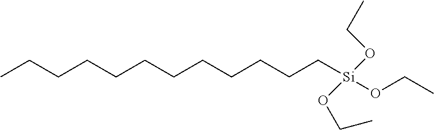 [SMILES: CCCCCCCCCCCC[Si](OCC)(OCC)OCC]

and octadecyltrimethoxysilane and/or octadecyltriethoxysilane.

In another preferred embodiment, a cosmetic agent is wherein it comprises at least one organic C₁-C₆ alkoxysilane of the formula (S-IV) selected from the group of
Methyltrimethoxysilane
Methyltriethoxysilane
Ethyltrimethoxysilane
Ethyltriethoxysilane
Propyltrimethoxysilane
Propyltriethoxysilane
Hexyltrimethoxysilane
Hexyltriethoxysilane
Octyltrimethoxysilane
Octyltriethoxysilane
Dodecyltrimethoxysilane,
Dodecyltriethoxysilane,
Octadecyltrimethoxysilane,
Octadecyltriethoxysilane,
their mixtures

and/or their condensation products.

It has been found that, regarding staining keratinous material, it is particularly preferable if the cosmetic agent comprises two structurally different alkoxysilanes.

In a preferred embodiment, a cosmetic agent is wherein it comprises at least one alkoxysilane of the formula (S-I) and at least one alkoxysilane of the formula (S-IV).

The corresponding hydrolysis or condensation products are, for example, the following compounds:

Hydrolysis of C₁-C₆ alkoxysilane of formula (S-I) with water (reaction scheme using 3-aminopropyltriethoxysilane as an example):

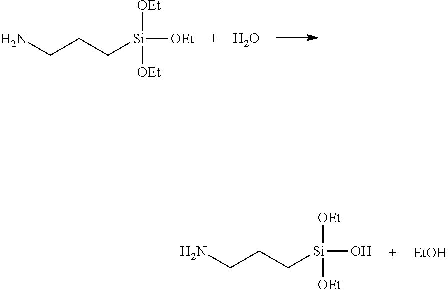 [SMILES: "EtOH" ; CCO[Si](CCCN)(OCC)OCC ; O ; CCO[Si](O)(CCCN)OCC]

Depending on the amount of water used, the hydrolysis reaction can also take place several times per C₁-C₆ alkoxysilane used:

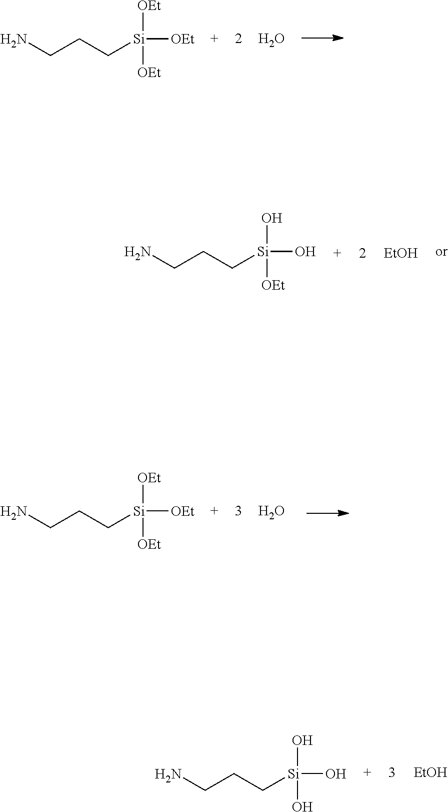

Hydrolysis of C₁-C₆ alkoxysilane of the formula (S-IV) with water (reaction scheme using methyltrimethoxysilane as an example):

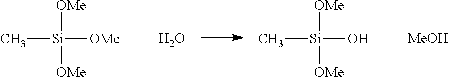

Depending on the amount of water used, the hydrolysis reaction can also take place several times per C₁-C₆ alkoxysilane used:

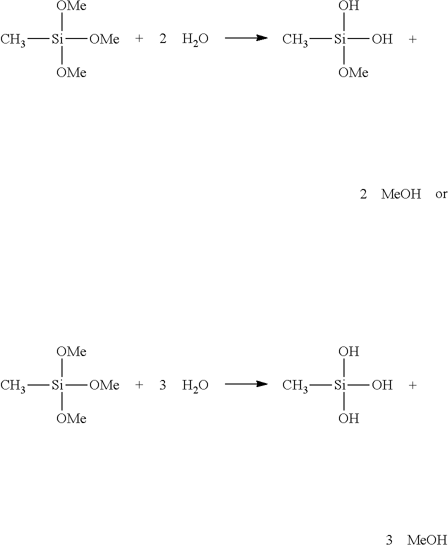

Condensation reactions include (shown using the mixture (3-aminopropyl)triethoxysilane and methyltrimethoxysilane):

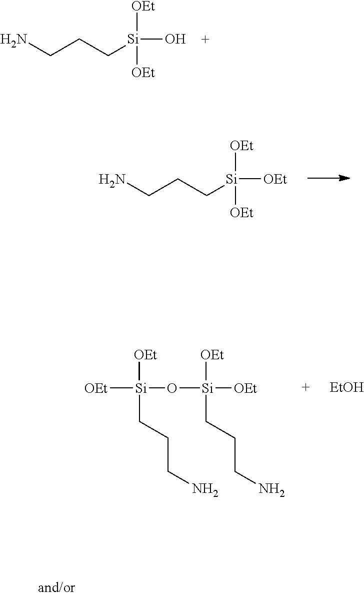

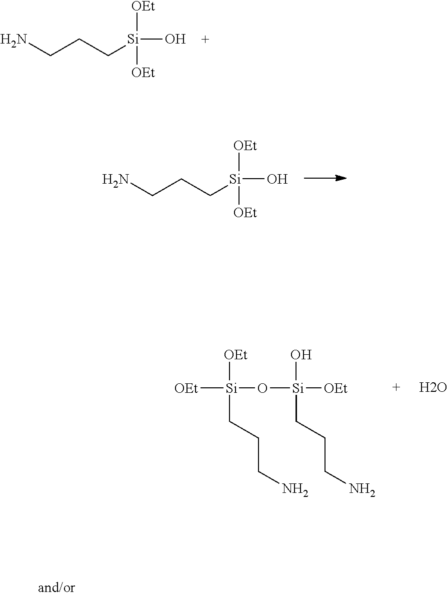 [SMILES: CCO[Si](O)(CCCN)OCC ; CCO[Si](O)(CCCN)OCC ; CCO[Si](O)(CCCN)O[Si](CCCN)(OCC)OCC ; O]

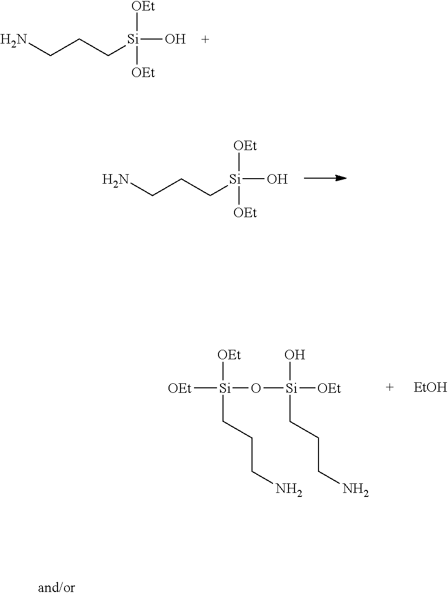

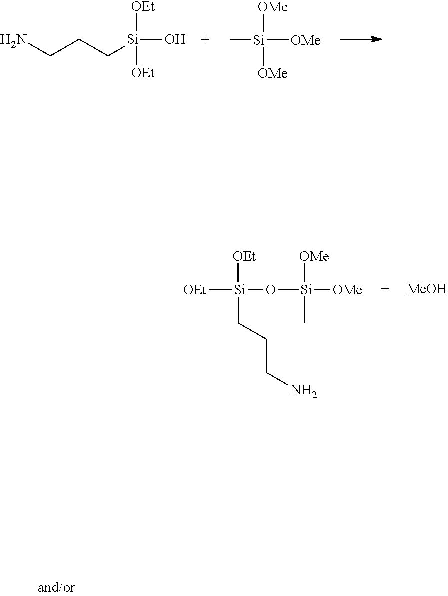

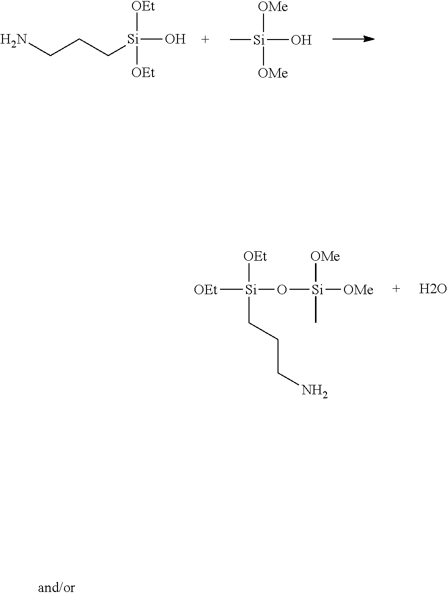

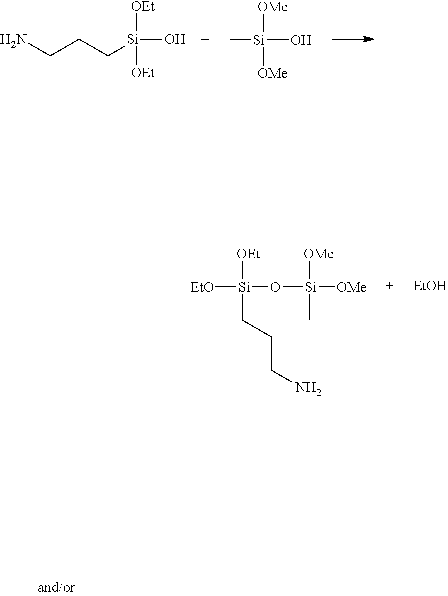

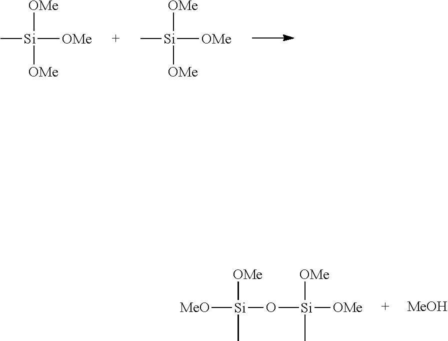

In the above exemplary reaction schemes the condensation to a dimer is shown in each case, but further condensations to oligomers with several silane atoms are also possible and preferred.

Both partially hydrolyzed and fully hydrolyzed C₁-C₆ alkoxysilanes of the formula (S-I) can participate in these condensation reactions, which undergo condensation with yet unreacted, partially or also fully hydrolyzed C₁-C₆ alkoxysilanes of the formula (S-I). In this case, the C₁-C₆ alkoxysilanes of formula (S-I) react with themselves.

Furthermore, both partially hydrolyzed and fully hydrolyzed C₁-C₆-alkoxysilanes of the formula (S-I) can also participate in the condensation reactions, which undergo condensation with not yet reacted, partially or also fully hydrolyzed C₁-C₆-alkoxysilanes of the formula (S-IV). In this case, the C₁-C₆ alkoxysilanes of formula (S-I) react with the C₁-C₆ alkoxysilanes of formula (S-IV).

Furthermore, both partially hydrolyzed and fully hydrolyzed C₁-C₆-alkoxysilanes of the formula (S-IV) can also participate in the condensation reactions, which undergo condensation with not yet reacted, partially or also fully hydrolyzed C₁-C₆-alkoxysilanes of the formula (S-IV). In this case, the C₁-C₆ alkoxysilanes of formula (S-IV) react with themselves.

The cosmetic agent may comprise one or more organic C₁-C₆ alkoxysilanes in various proportions. This is determined by the specialist depending on the desired application. In the case of coloring keratinous material, for example, the amount may depend on the thickness of the silane coating on the keratinous material and the amount of keratinous material to be treated.

Particularly storage-stable cosmetic agents with very good coloring results when applied to keratinous material could be obtained when the cosmetic agent comprises—based on its total weight—one or more organic C₁-C₆ alkoxysilanes and/or the condensation products thereof in a total amount of from about 30 to about 85 wt. %, preferably from about 35 to about 80 wt. %, more preferably from about 40 to about 75 wt. %, still more preferably from about 45 to about 70 wt. % and very particularly preferably from about 50 to about 65 wt. %.

It may be preferred that the cosmetic agent ready for use comprises further ingredients, in particular water, in addition to the pigment suspension as contemplated herein and the organic C₁-C₆ alkoxysilane(s).

The cosmetic agent comprises alkoxysilanes, a class of highly reactive compounds that can undergo hydrolysis or oligomerization and/or polymerization when applied. To avoid premature oligomerization or polymerization, it may be of significant advantage to the user to prepare the ready-to-use cosmetic agent just prior to application.

To increase user convenience, the user is preferably provided with all the necessary agents in the form of a multi-component packaging unit (kit-of-parts).

Thus, a third object of the present disclosure is a multi-component packaging unit (kit-of-parts) comprising, separately assembled from each other
a first container comprising an agent (a′), wherein the agent comprises (a′):
   (a1) at least one or more organic C₁-C₆-alkoxysilanes, and
a second container comprising an agent (a″), wherein the agent comprises (a″):
   (a2) a pigment suspension as contemplated herein.

Regarding further preferred embodiments of the cosmetic agent and/or the multicomponent packaging unit (kit-of-parts), what has been said about the pigment suspensions applies mutatis mutandis.

### Examples

The following formulations have been produced (unless otherwise indicated, all figures are in wt. %)

Agent (a′)

| | wt.-% |
|---|---|
| (3-Aminopropyl)tri ethoxy silane | 24 |
| Methyltriethoxysilane | 72 |
| Water | ad 100 |

Agent (a″) (= pigment suspension)

| | Wt.% |
|---|---|
| Pigment mixture (CI 12490, CI 74160 and CI 11680) | 5 |
| Triethyl citrate | ad 100 |

Cosmetic agent (a) was prepared by mixing 5 g of agent (a′) and 5 g of agent (a″).

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A pigment suspension, comprising:
(a) at least one coloring compound selected from the group of pigments; and
(b) a hydroxycarboxylic acid ester.

2. The pigment suspension of claim 1, wherein the at least one coloring compound (a) comprises a pigment comprising a substrate platelet, and wherein the substrate platelet comprises a metal, a metal alloy, natural mica, or a synthetic mica.

3. The pigment suspension of claim 2, wherein the substrate platelet comprises a metal selected from the group of aluminum, copper, silver, and gold.

4. The pigment suspension of claim 2, wherein the substrate platelet comprises a metal alloy comprising brass.

5. The pigment suspension of claim 2, wherein the substrate platelet comprises synthetic mica (INCI: Synthetic Fluorophlogopite).

6. The pigment suspension of claim 1, wherein the at least one coloring compound (a) comprises at least one organic pigment selected from the group of carmine, quinacridone, phthalocyanine, sorghum, blue pigments having the Color Index numbers CI 42090, CI 69800, CI 69825, CI 73000, CI 74100, CI 74160, yellow pigments having the Color Index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, CI 75470 and mixtures thereof.

7. The pigment suspension of claim 1, wherein the hydroxycarboxylic acid ester (b) comprises citric acid triethyl ester.

8. A cosmetic agent, comprising:
(I) a pigment suspension comprising:
(a) at least one coloring compound selected from the group of pigments, and
(b) a hydroxycarboxylic acid ester; and
(II) one or more organic C₁-C₆ alkoxysilanes and/or condensation products thereof.

9. The cosmetic agent of claim 8, wherein the one or more organic C₁-C₆ alkoxysilanes and/or condensation products thereof (II) comprises an alkoxysilane of the formula (S-I) and/or (S-II) and/or (S-IV):
R₁R₂N-L-Si(OR₃)ₐ(R₄)_{b} (S-I),
wherein in formula (S-I),
R₁ and, R₂ each independently represent a hydrogen atom or a C₁-C₆ alkyl group,
L is a linear or branched divalent C₁-C₂₀ alkylene group,
each R₃ and; R₄ independently represents a C₁-C₆ alkyl group,
a is an integer of from 1 to 3, and
b is the difference of 3-a;
(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (S-II),
wherein in formula (S-II),
each R₅, R₅′, R₅″, R₆, R₆′, and R₆″ independently represents a C₁-C₆ alkyl group,
each A, A′, A″, A′″, and A″″ independently represents a linear or branched divalent C₁-C₂₀ alkylene group,
each R₇ and R₈ independently represents a hydrogen atom, a C₁-C₆ alkyl group, a hydroxy C₁-C₆ alkyl group, a C₂-C₆ alkenyl group, an amino C₁-C₆ alkyl group or a group of formula (S-III);
-(A″″)—Si(R₆″)_{d″}(OR₅″)_{c″} (S-III)
c, c′, and c″ each independently represent an integer of from 1 to 3,
d is the difference of 3-c,
d′ is the difference of 3-c′,
d″ is the difference of 3-c″,
e, f, g, and h are each independently 0 or 1,
provided that at least one of e, f, g and h are different from 0;
R₉Si(OR₁₀)ₖ(R₁₁)ₘ (S-IV),
wherein in formula (S-IV),
R₉ is a C₁-C₁₂ alkyl group,
each —R₁₀ and R₁₁ independently represents a C₁-C₆ alkyl group,
k is an integer of from 1 to 3, and
m is the difference of 3-k.

10. The cosmetic agent of claim 9, wherein the one or more organic C₁-C₆ alkoxysilanes and/or condensation products thereof (II) comprises at least two structurally different organic C₁-C₆ alkoxysilanes each of formula (S-I) and/or (S-II) and/or (S-IV).

11. A multi-component packaging unit (kit-of-parts) for dyeing keratinous material, comprising, separately packaged:
a first container comprising an agent (a′), wherein the agent (a′) comprises
(a1) one or more organic C₁-C₆-alkoxysilanes and/or condensation products thereof; and
a second container comprising an agent (a″), wherein the agent (a″) comprises (a2) the pigment suspension of claim 1.

12. The multi-component packaging unit of claim 11, wherein in the pigment suspension (a2), the at least one coloring compound (a) comprises a pigment, where the pigment comprises a substrate platelet comprising aluminum, copper, silver, and/or gold.

13. The multi-component packaging unit of claim 11, wherein in the pigment suspension (a2), the at least one coloring compound (a) comprises a pigment, where the pigment comprises a substrate platelet comprising a brass alloy.

14. The multi-component packaging unit of claim 11, wherein in the pigment suspension (a2), the at least one coloring compound (a) comprises a pigment, where the pigment comprises a synthetic mica (INCI: Synthetic Fluorophlogopite).

15. The multi-component packaging unit of claim 11, wherein in the pigment suspension (a2), the hydroxycarboxylic acid ester (b) comprises citric acid triethyl ester.

16. The cosmetic agent of claim 8, wherein in the pigment suspension (I), the at least one coloring compound (a) comprises a pigment, where the pigment comprises a substrate platelet comprising aluminum, copper, silver, and/or gold.

17. The cosmetic agent of claim 8, wherein in the pigment suspension (I), the at least one coloring compound (a) comprises a pigment, where the pigment comprises a substrate platelet comprising a brass alloy.

18. The cosmetic agent of claim 8, wherein in the pigment suspension (I), the at least one coloring compound (a) comprises a pigment, where the pigment comprises a synthetic mica (INCI: Synthetic Fluorophlogopite).

19. The cosmetic agent of claim 8, wherein in the pigment suspension (I), the at least one coloring compound (a) comprises at least one organic pigment selected from the group of carmine, quinacridone, phthalocyanine, sorghum, blue pigments having the Color Index numbers CI 42090, CI 69800, CI 69825, CI 73000, CI 74100, CI 74160, yellow pigments having the Color Index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, CI 75470, and mixtures thereof.

20. The cosmetic agent of claim 8, wherein in the pigment suspension (I), the hydroxycarboxylic acid ester (b) comprises citric acid triethyl ester.
